(19) United States
(12) Patent Application Publication
(10) Pub. No.: US 2015/0143633 A1
(43) Pub. Date: May 28, 2015

(54) Patient Support Apparatus and Medical Imaging Apparatus having the Patient Support Apparatus

(71) Applicants: Wolfgang Kraus, Fuerth (DE); Thomas Kundner, Buckenhof (DE)

(72) Inventors: Wolfgang Kraus, Fuerth (DE); Thomas Kundner, Buckenhof (DE)

(21) Appl. No.: 14/555,674

(22) Filed: Nov. 27, 2014

(30) Foreign Application Priority Data
Nov. 27, 2013 (DE) 102013224267.0

(51) Int. Cl. A61B 5/055; A61G 13/12

(52) U.S. Cl. CPC A61B 5/0555; A61G 13/12
USPC 5/601

(57) ABSTRACT

A patient support apparatus configured to position and/or support a patient on a patient couch is provided. The patient support apparatus includes a first support unit that has a support surface to support the patient directly. The patient support apparatus also includes a second support unit. The second support unit includes at least one filler and a receiving chamber for receiving the at least one filler. The at least one filler includes a flexibly adjustable filler.

This application claims the benefit of DE 10 2013 224 267.0, filed on Nov. 27, 2013, which is hereby incorporated by reference in its entirety.

### BACKGROUND

When supporting and/or positioning a patient on a patient couch for a medical examination using imaging, the problem frequently arises that it is not possible or it is not possible to an adequate degree to support individual regions of the patient (e.g., an arm or leg of the patient) in a convenient and/or comfortable manner. Additional units (e.g., local coils for magnetic resonance examinations and/or connectors, etc.) also make it difficult to position the patient in a convenient manner.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, a patient is supported in a particularly convenient and comfortable manner on a patient couch for a medical examination using imaging.

In one embodiment, a patient support apparatus configured to position and/or support a patient on a patient couch (e.g., having a first support unit that has a support surface to support the patient directly) is provided.

The patient support apparatus includes a second support unit. The second support unit includes at least one filler and a receiving chamber for receiving the at least one filler. The at least one filler includes a flexibly adjustable filler. In this context, a flexibly adjustable filler refers, for example, to a filler that may be shaped in an elastic manner as a whole and, for example, when subject to the influence of a force acting on the filler. The filler may, for example, be a flexibly adjustable material (e.g., one that may be shaped in an elastic manner) and/or may include a number of individual filler elements. In one embodiment, the individual filler elements may be supported such that the individual filler elements may be displaced in relation to one another within the second support unit, so that the second support unit (e.g., the filler of the second support unit) may have different contours to compensate for irregularities in a support surface provided to support the patient support apparatus. This allows the provision of a particularly adjustable patient support apparatus for supporting and/or positioning the patient. The patient support apparatus (e.g., the second support unit) may, for example, be adjusted for a bearing surface and/or a base of the patient couch, so that there is always a flat support surface to support the patient. The second support unit (e.g., just the second support unit) may be configured to compensate for irregularities relating to the bearing of the patient support apparatus.

In one embodiment, the filler of the second support unit has a plurality of individual filler elements that may be moved in relation to one another. The second support unit (e.g., the plurality of individual filler elements that may be moved in relation to one another) may be used to compensate for different contours of a bearing surface (e.g., of a patient couch) for supporting the patient support apparatus, so that there is always a flat and regular support surface for the patient. In one embodiment, the second support unit may take on different volumes for this purpose. This allows the provision of a particularly adjustable patient support apparatus that may be used in a flexible manner to support and/or position the patient.

An economical patient support apparatus may be provided if the filler of the second support unit includes polystyrene balls and/or a flocked foam. A particularly light and weight-saving patient support apparatus may thus be provided. Alternatively or additionally, the filler may also be a gel and/or a gas and/or further fillers with a flexibly adjustable volume.

In one development, the receiving chamber of the second support unit has at least one cutout for the passage of cables. Such a patient support apparatus may be used for a medical examination using imaging (e.g., a medical magnetic resonance examination). Cables from further units, such as local coils, ECG unit, etc., that are arranged on the patient couch for the medical examination using imaging (e.g., a medical magnetic resonance examination) are already dealt with by the at least one cutout when the patient is supported in a flat and comfortable manner. In one embodiment, interference with or damage to the cables passed on the patient couch using the patient support apparatus may be prevented.

In one embodiment, the receiving chamber of the second support unit has a sheath that has a support surface to support and/or position at least one subregion of the patient. This allows the patient support apparatus to be used in a flexible manner, in that the first support unit or the second support unit may be used to support and/or position the patient (e.g., subregions of the patient). For example, the patient support apparatus may be used such that the second support unit has direct contact with the patient to brace and/or support arms or legs of patients in order to achieve a high level of flexibility and adjustability when positioning in an examination position.

The receiving chamber of the second support unit may also have an opening with a closing unit so that the fill level of the filler within the receiving chamber may be varied and adjusted for an examination situation. For example, the fill level of the filler within the receiving chamber may be adjusted for an application and/or a base (e.g., the patient couch), thereby advantageously increasing the flexibility of the patient support apparatus. The closing unit may include a valve element and/or a pump, so that medical personnel may operate the patient support apparatus in a particularly simple manner.

In a further embodiment, the first support unit has a filler. The filler includes, for example, a single filler element, so that a stable and regular surface may be achieved for supporting and/or positioning the patient. For example, the filler includes a polyurethane foam (PUR foam). The filler of the first support unit is, for example, configured so that the filler is less flexibly adjustable than the filler of the second support unit. In one embodiment, the different firmnesses and/or the different hardnesses of the fillers may be used to optimize the flexibility of the patient support apparatus with respect to both adjustment for a substrate and/or a base for supporting the patient support apparatus, and also with respect to a convenient (e.g., a flat and regular) support surface for the patient.

A particularly stable and regular (e.g., flat) support surface for the patient may be provided if the first support unit has a stabilizing element. This allows a flat support surface to be provided in a structurally simple manner to support and/or position the patient. Also, the support surface may be embodied in a flat and regular manner independently of a bearing surface and/or a base for the patient support apparatus, as irregularities in a bearing surface and/or base for the patient support apparatus may be compensated for by the second support unit. The support surface of the first support unit may be decoupled from the second support unit (e.g., a contour of the second support unit, by means of the stabilizing element. In one embodiment, adjustment and/or sagging of the first support unit and therefore also of the first support surface may be provided. The stabilizing element may include, for example, a wooden plate and/or a plastic plate and/or further embodiments of the stabilizing element.

If the stabilizing element is configured as a single piece with the filler element, a particularly economical patient support apparatus may be provided. For example, the filler element may, for example, include a very solid foam that has a compression hardness that is greater than a compression hardness of the at least one filler of the second support unit. A compression hardness of the filler element of the first support unit, which is configured as a single piece with the stabilizing element, is to be configured such that when the patient is positioned on the patient support apparatus, any sagging of the first support unit and/or adjustment of the first support unit to a bearing surface for bearing the patient support apparatus is prevented. A single piece may, for example, be formed as one part and/or from one component.

A particularly high level of comfort may be achieved for the patient lying down if the first support unit has a sheath. The sheath may enclose a receiving region of the first support unit for receiving the filler element and/or the stabilizing element. For example, the sheath of the first support unit includes a synthetic leather so that the first support unit (e.g., the sheath) may be cleaned in a particularly simple manner, thereby allowing a high standard of hygiene to be achieved for the patient support apparatus.

In a further embodiment, the sheath of the first support unit and the sheath of the second support unit are configured at least partially as a single piece, thereby allowing a particularly compact patient support apparatus to be provided. For example, the first support unit may directly adjoin the second support unit, so that the first support unit and the second support unit have a shared separating wall. The shared separating wall includes both the sheath of the first support unit and the sheath of the second support unit and is arranged between the first support unit and the second support unit.

In one embodiment, the patient support apparatus includes a multi-layered structure. The multi-layered structure includes at least one first layer and at least one second layer. The at least one first layer includes the first support unit, and the at least one second layer includes the second support unit. A particularly compact patient support apparatus may be provided, which achieves an essentially decoupled functionality of the individual layers (e.g., the individual support units).

A use of the patient support apparatus together with a magnetic resonance apparatus may be achieved if the first support unit and/or the second support unit is/are configured to be magnetic resonance-compatible.

In one embodiment, a patient couch having a table and a patient support apparatus arranged on the table is provided. The patient support apparatus is configured to position and/or support a patient on the patient couch. For example, the patient support apparatus includes a first support unit that has a support surface to support the patient directly, and a second support unit. The second support unit includes at least one filler and a receiving chamber for receiving the at least one filler. The at least one filler includes a flexibly adjustable filler. This allows the patient to be positioned and/or supported on the patient couch in an adjustable manner by the patient support apparatus. For example, the second support unit (e.g., the filler of the second support unit) may be used to compensate for irregularities in a support surface provided to support the patient support apparatus, thereby allowing a regularly flat support surface for supporting and/or positioning the patient to be provided in a simple manner.

In one embodiment, a medical imaging apparatus having a patient couch and a patient support apparatus configured to position and/or support a patient on a patient couch is provided. For example, the patient support apparatus includes a first support unit that has a support surface to support the patient directly, and a second support unit. The second support unit includes at least one filler and a receiving chamber for receiving the at least one filler. The at least one filler includes a flexibly adjustable filler. The advantages of the patient couch and the medical imaging apparatus correspond essentially to the advantages of the patient support apparatus, which are set out in detail above. Features, advantages or alternative embodiments mentioned in this context may also be applied to the other subject and vice versa.

### BRIEF DESCRIPTION OF THE DRAWINGS

FIG. 1 shows a schematic diagram of one embodiment of a medical imaging apparatus having a patient couch including a patient support apparatus;

FIG. 2 shows a schematic sectional diagram of one embodiment of the patient support apparatus; and

FIG. 3 shows a schematic sectional diagram of an alternative embodiment of the patient support apparatus.

### DETAILED DESCRIPTION

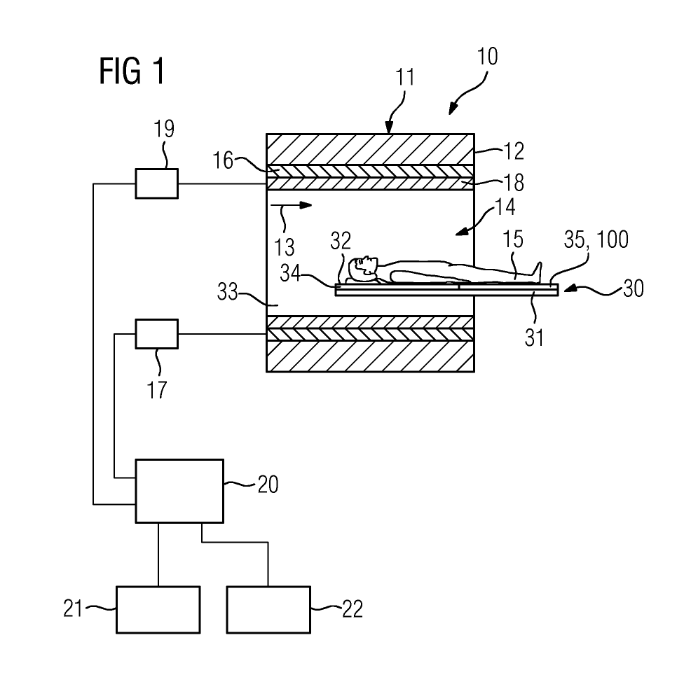

FIG. 1 shows a schematic diagram of one embodiment of a medical imaging apparatus 10 formed by a magnetic resonance apparatus. Alternatively, the medical imaging apparatus 10 may be a computed tomography apparatus, a PET apparatus, a C-arm apparatus and/or further configurations of a medical imaging apparatus 10.

The magnetic resonance apparatus includes a magnet unit 11 having a main magnet 12 for generating a powerful and, for example, constant main magnetic field 13. The magnetic resonance apparatus also includes a patient receiving region 14 for receiving a patient 15. In the present exemplary embodiment, the patient receiving region 14 is configured in a cylindrical shape and is enclosed in a cylindrical manner in a circumferential direction by the magnet unit 11. However, a different configuration of the patient receiving region 14 from this may be provided. The patient 15 may be moved into the patient receiving region 14 by a patient couch 30 of the magnetic resonance apparatus.

The magnet unit 11 also includes a gradient coil unit 16 for generating magnetic field gradients used for spatial encoding during imaging. The gradient coil unit 16 is controlled by a gradient control unit 17 of the magnetic resonance apparatus. The magnet unit 11 further includes a high-frequency antenna unit 18 and a high-frequency antenna control unit 19 for exciting a polarization, which becomes established in the main magnetic field 13 generated by the main magnet 12. The high-frequency antenna unit 18 is controlled by the high-frequency antenna control unit 19 and emits high-frequency magnetic resonance sequences into an examination chamber essentially formed by the patient receiving region 14.

To control the main magnet 12 of the gradient control unit 17 and to control the high-frequency antenna control unit 19, the magnetic resonance apparatus includes a control unit 20 formed by a computation unit. The control unit 20 controls the magnetic resonance apparatus centrally (e.g., with the performance of a predetermined imaging gradient echo sequence). The control unit 20 also includes an evaluation unit (not shown in detail) for evaluating image data. Control information (e.g., imaging parameters) and reconstructed magnetic resonance images may be displayed to an operator on a display unit 21 (e.g., on at least one monitor) of the magnetic resonance apparatus. The magnetic resonance apparatus also includes an input unit 22, by which an operator may input information and/or parameters during a measuring operation.

The patient couch 30 has a table 31 with a bearing surface 32 for supporting and/or positioning the patient 15. The table 31 is supported in a movable manner so that a patient 15 may be introduced into the patient receiving region 14 of the magnetic resonance apparatus using the table 31. The patient couch 30 also includes a base unit 33. The table 31 is supported such that the table 31 may be moved in relation to the base unit 33. The patient couch 31 includes a sliding bearing unit (not shown in detail) to support the table 31 such that the support table 31 may be moved in relation to the base unit 33. In the present exemplary embodiment, the base unit 33 is integrated within a housing of the magnet unit that encloses the patient receiving region 14.

The bearing surface 32 of the table 31 (e.g., an embodiment of the bearing surface 32) depends on an upcoming magnetic resonance examination of the patient 15, as different support surface units 34 are used for different magnetic resonance examinations. High-frequency receive antenna units may be already integrated in the support surface units 34. For example, the subregion of the bearing surface, on which the region of the patient 15 to be examined comes to rest, is equipped with a support surface unit 34 that includes a high-frequency receive antenna unit.

In order to provide a flat and convenient support surface to support the patient 15 for medical examinations using imaging (e.g., for magnetic resonance examinations), the patient couch 30 also includes a patient support apparatus 35. The patient support apparatus 35 is configured to position and/or support the patient 15 on the patient couch 30. To support and/or position the patient 15 on the patient couch 30 (e.g., on the table 31), the patient support apparatus 35 is arranged between the patient 15 and the table 31, so that the patient 15 may be supported in a comfortable and convenient manner regardless of the embodiment of the table 31. For example, the patient support apparatus 35 is arranged in the regions of the table 31, in which there is no support surface unit 34, so that there is also compensation between the individual regions of the table 31 with and without a support surface unit 34 using the patient support apparatus 35.

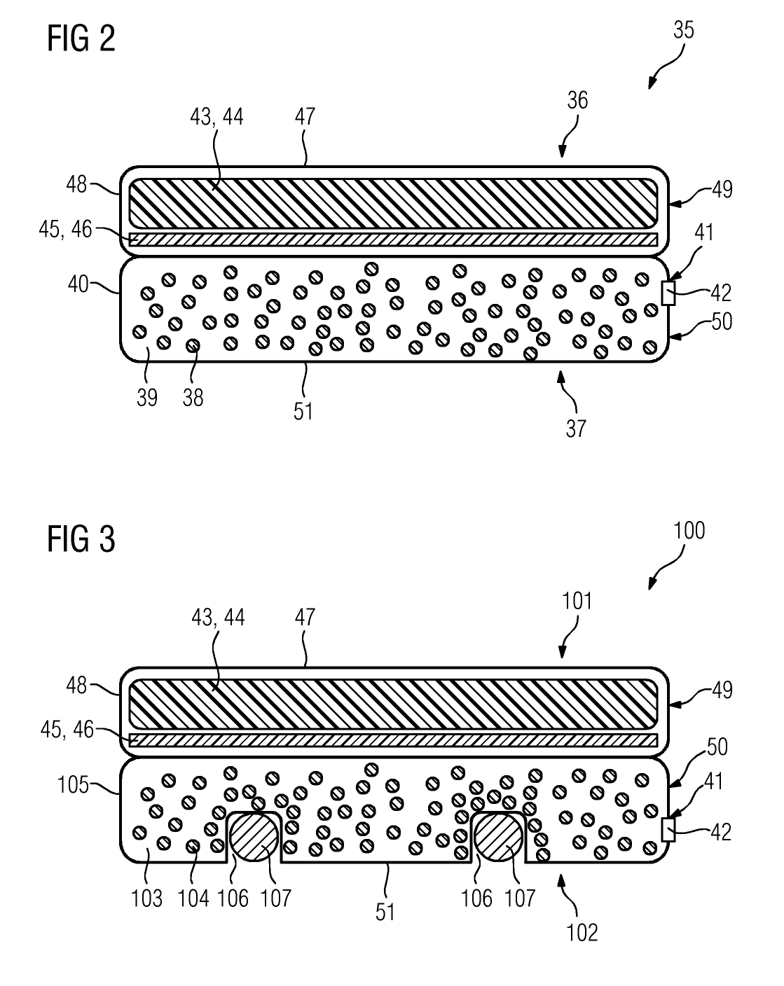

The patient support apparatus 35 is shown in more detail in FIG. 2 and has a first support unit 36 and a second support unit 37. The second support unit 37 has a filler 38 and a receiving chamber 39 for receiving the filler 38. The filler 38 includes a flexibly adjustable filler 38. In the present exemplary embodiment, the filler 38 of the second support unit 37 includes a plurality of individual filler elements that may be moved in relation to one another and in the present instance, by way of example, are formed at least partially by polystyrene balls and/or a flocked foam. The configuration of the filler 38 of the second support unit 37 is, however, not limited to polystyrene balls and/or a flocked foam and/or a plurality of individual filler elements that may be moved in relation to one another. The filler 38 of the second support unit 37 may also be, for example, a gel and/or a gas and/or a liquid and/or further fillers 38. The filler 38 of the second support unit 37 may be tailored for an application of the patient support apparatus 35 and/or the patient couch 30.

The second support unit 37 (e.g., the flexible adjustable filler 38 of the second support unit 37) may be used to adjust the patient support apparatus 35 for different contours of the patient couch 30 (e.g., of the table 31).

The second support unit 37 (e.g., the receiving chamber 39) also has a sheath 40 in FIG. 2. The sheath 40 may be formed, for example, at least partially from a synthetic leather, so that cleaning of the sheath 40 may be provided. The sheath 40 may include any materials that allow easy cleaning of the second support unit 37.

The receiving chamber 39 of the second support unit 37 also includes an opening 41 with a closing unit 42. The opening 41 with the closing unit 42 may be used for the feeding in and/or topping up of the filler 38. For example, when the filler 38 is configured as a gel and/or gas and/or liquid, the closing unit 42 may include a valve unit and/or a pump. Alternatively, the patient support apparatus 35 (e.g., the second support unit 38) may also be configured without an opening 41 and therefore also without a closing unit 42. This may be provided when the filler 38 is configured as polystyrene balls and/or flocked foam.

The first support unit 36 of the patient support apparatus 35 has a layered structure and has a first layer 43 that includes a filler 44 of the first support unit 36. The filler 44 of the first support unit 36 is, for example, configured to be harder than the filler 38 of the second support unit 37. The filler 44 of the first support unit 36 is, for example, embodied so that the filler 44 is less flexibly adjustable than the filler 38 of the second support unit 37. The filler 44 of the first support unit 36 includes, for example, a single filler element, providing a stable and, for example, regular surface for supporting and/or positioning the patient 15. In the present embodiment, the filler 44 of the first support unit 36 includes a PUR foam.

The first support unit 36 also includes a stabilizing element 45 that includes a second layer 46 of the first support unit 36. The stabilizing element 45 may include, for example, a wooden plate and/or a plastic plate and/or further embodiments of the stabilizing element 45. The stabilizing element 45 allows a regular and flat support surface 47 to be achieved for the patient support apparatus 35 and advantageously prevents adjustment of a support surface 47 of the patient support apparatus 35 for a contour of a bearing surface of the patient couch 30 for the patient support apparatus 35. The stabilizing element 45 may also prevent sagging of the first support unit 36 when the patient 15 is loaded on the patient support apparatus 35.

In an alternative embodiment of the first support unit 36, the stabilizing element 45 and the filler 44 of the first support unit 36 may be configured as a single piece with one another.

The first support unit 36 also has a sheath 48 that encloses the filler 44 of the first support unit 36 and the stabilizing element 45. The sheath 48 of the second support unit 36, for example, includes a synthetic leather, so that it is particularly easy to clean the first support unit 36 (e.g., the sheath 48), thereby allowing a high standard of hygiene to be achieved for the patient support apparatus 35.

The first support unit 36 and the second support unit 37 provide that the patient support apparatus 35 has a multi-layered structure. The multi-layered structure includes a first layer 49 and a second layer 50 in one embodiment. The first layer 49 includes, for example, the first support unit 36, and the second layer 50 includes the second support unit 37.

The two layers 49, 50 (e.g., the two support units 36, 37) are also arranged to that the two layers 49, 50 adjoin one another directly. A side of the sheath 48 of the first support unit 36 facing the second support unit 37 is configured as a single piece with a side of the sheath 40 of the second support unit 37 facing the first support unit 36.

In one alternative embodiment, the multi-layered structure of the patient support apparatus 35 may have more than two layers 49, 50 and/or more than two support units 36, 37. For example, the stabilizing element 45 may be arranged separately from the filler 44 of the first support unit 36 in a separate support unit.

The sheath 48 of the first support unit 36 has a support surface 47 to support the patient 15 directly. The support surface 47 of the first support unit 36 is arranged on a side of the first support unit 36 facing away from the second support unit 37.

The sheath 40 of the second support unit 37 also has a support surface 51 that is arranged on a side of the second support unit 37 facing away from the first support unit 36. The support surface 51 of the second support unit 37 may also be used to support subregions of the patient 15 in that the first support surface 47 of the first support unit 36 serves to bear on a base against the patient couch 30. In such an application, there may already be a flat base to bear the patient support apparatus 53. The patient support apparatus 35 includes, for example, the function of specifically supporting and/or positioning subregions of the patient 15 (e.g., arranging a knee of the patient 15) in a desired position (e.g., in a bent position) for the medical examination using imaging.

In the present exemplary embodiment, the patient support apparatus 35 is also configured to be magnetic resonance-compatible, so that the patient support apparatus 35 may be deployed and/or used for a magnetic resonance examination.

FIG. 3 shows an alternative exemplary embodiment of the patient support apparatus 100. Components, features and functions that remain essentially same are shown with same reference characters. The description that follows is limited essentially to the differences compared with the exemplary embodiment in FIG. 2, with reference being made to the description of the exemplary embodiment in FIG. 2 with respect to components, features and functions that remain the same.

FIG. 3 shows one embodiment of a patient support apparatus 100 embodied in an alternative manner to the one in FIG. 2. The patient support apparatus 100 includes a first support unit 101 and a second support unit 102.

The embodiment and configuration of the first support unit 101 correspond to the details relating to the exemplary embodiment in FIG. 2.

The second support unit 102 includes a receiving chamber 103, a filler 104 and a sheath 105. The filler 104 and the sheath 105 are configured in an analogous manner as set out in the details relating to the exemplary embodiment in FIG. 2. The receiving chamber 103 in FIG. 3 also includes cutouts 106 configured for the passage of cables 107 from, for example, the patient couch 20 and/or from additional units for the medical examination using imaging (e.g., an injection unit and/or an ECG unit). This prevents the cables from being squashed. The number and spatial configuration and/or arrangement of the cutouts 106 may, however, differ from the present embodiments.

The illustrated patient support apparatus 35 and the illustrated magnetic resonance apparatus may include further components that are standard for patient support apparatuses 35 and magnetic resonance apparatuses. A general mode of operation of a magnetic resonance apparatus and patient support apparatus 35 is also known to the person skilled in the art so there is no need for a detailed description of the general components.

Although the invention has been illustrated and described in detail using the exemplary embodiments, the invention is not restricted by the disclosed examples, and other variations may be derived therefrom by the person skilled in the art without departing from the scope of protection of the invention.

It is to be understood that the elements and features recited in the appended claims may be combined in different ways to produce new claims that likewise fall within the scope of the present invention. Thus, whereas the dependent claims appended below depend from only a single independent or dependent claim, it is to be understood that these dependent claims can, alternatively, be made to depend in the alternative from any preceding or following claim, whether independent or dependent, and that such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A patient support apparatus configured to position, support, or position and support a patient on a patient couch, the patient support apparatus comprising:
a first support unit that has a support surface to support the patient directly; and
a second support unit comprising at least one filler and a receiving chamber for receiving the at least one filler, the at least one filler comprising a flexibly adjustable filler.

2. The patient support apparatus of claim 1, wherein the filler of the second support unit comprises a plurality of individual filler elements that are movable in relation to one another.

3. The patient support apparatus of claim 1, wherein the filler of the second support unit comprises polystyrene balls, a flocked foam, or the polystyrene balls and the flocked foam.

4. The patient support apparatus of claim 1, wherein the receiving chamber of the second support unit has at least one cutout for the passage of cables.

5. The patient support apparatus of claim 1, wherein the receiving chamber of the second support unit has a sheath, the sheath having a support surface to support, position, or support and position at least one subregion of the patient.

6. The patient support apparatus of claim 1, wherein the receiving chamber of the second support unit has an opening with a closing unit.

7. The patient support apparatus of claim 1, wherein the first support unit has a filler.

8. The patient support apparatus of claim 7, wherein the first support unit has a stabilizing element.

9. The patient support apparatus of claim 8, wherein the stabilizing element is configured as a single piece with the filler element.

10. The patient support apparatus of claim 5, wherein the first support unit has a sheath.

11. The patient support apparatus of claim 10, wherein the sheath of the first support unit and the sheath of the second support unit are configured at least partially as a single piece.

12. The patient support apparatus of claim 1,
further comprising a multi-layered structure, the multi-layered structure having at least one first layer and at least one second layer, the at least one first layer comprising the first support unit, and the at least one second layer comprising the second support unit.

13. The patient support apparatus of claim 1,
wherein the first support unit, the second support unit, or the first support unit and the second support unit are configured to be magnetic resonance-compatible.

14. A patient couch comprising:
a table; and
a patient support apparatus configured to position, support, or position and support a patient on the patient couch, the patient support apparatus comprising:
a first support unit that has a support surface to support the patient directly; and
a second support unit comprising at least one filler and a receiving chamber for receiving the at least one filler, the at least one filler comprising a flexibly adjustable filler.

15. A medical imaging apparatus comprising:
a patient couch; and
a patient support apparatus configured to position, support, or position and support a patient on a patient couch, the patient support apparatus comprising:
a first support unit that has a support surface to support the patient directly; and
a second support unit comprising at least one filler and a receiving chamber for receiving the at least one filler, the at least one filler comprising a flexibly adjustable filler.
